(19) United States
(12) Patent Application Publication
(10) Pub. No.: US 2020/0316266 A1
(43) Pub. Date: Oct. 8, 2020

(54) SILICON-REINFORCED VEIN STENT

(71) Applicant: Kalie Christine Brown, Lovington, NM (US)

(72) Inventor: Kalie Christine Brown, Lovington, NM (US)

(21) Appl. No.: 16/839,901

(22) Filed: Apr. 3, 2020

Related U.S. Application Data
(60) Provisional application No. 62/828,486, filed on Apr. 3, 2019.

(51) Int. Cl. A61L 31/08; A61F 2/90; A61L 31/02; A61L 31/14

(52) U.S. Cl. CPC A61L 31/088; A61F 2/90; A61F 2/958; A61L 31/14; A61F 2210/0014; A61L 31/022

(57) ABSTRACT

The silicon-reinforced stent is a specially designed stent structured and arranged with a silicon coating over the metal stent structure to maintain a firmer and longer lasting stent. The silicon may be similar to Vaseline and may also prevent plaque from further accumulating in the vein. Additionally, the silicon coating will help prevent the edges of the metal structure from collapsing, thereby ensuring that if the stent moves or comes out, the movement will cause less damage.

### CROSS-REFERENCE TO RELATED APPLICATION

The present application is related to and claims priority from prior provisional application Ser. No. 62/828,486, filed Apr. 3, 2019 which application is incorporated herein by reference.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

### 1. Field of the Invention

The presently claimed invention relates generally to the field of stent devices and more specifically relates to a reinforced vein coating structured and arranged for applying to the stent to maintain a firmer and longer lasting stent.

### 2. Description of the Related Art

In medicine, a stent is a metal or plastic tube inserted into the lumen of an anatomic vessel or duct to keep the passageway open. There is a wide variety of stents used for different purposes, from expandable coronary and vascular and biliary stents to simple plastic stents used to allow the flow of urine between kidney and bladder. “Stent” is also used as a verb to describe the placement of such a device, particularly when a disease such as atherosclerosis has pathologically narrowed a structure, such as an artery, although the placement of a stent may also be referred to as “stenting.”

Typically, expandable stents are usually a mesh-like tube made from a flexible metal, such nickel titanium, commonly know as nitinol. However, as a bare-metal vascular stents can lead to thrombosis, or inflammation scarring, drug-eluting stents have also been developed to help counteract this and serve as drug delivery vehicles. Another common type of stent is a stent graft or covered stent, where the metal mesh is covered with a fabric coating to create a contained tube that is still expandable.

If a stent becomes dislodged, or even moves, it can cause injury, including a heart attack. Currently available vein stent designs are soft and don't last very long, requiring additional later surgeries. Currently available stents are also prone to plaque build-up, which can cause the stent to collapse over time.

Various attempts have been made to solve problems found in stent devices art. Among these are found in: U.S. Pat. No. 9,393,352 to Huang et al.; U.S. Pat. No. 6,635,078 to Zhong et al.; U.S. Pat. No. 8,535,372 to Fox et al.; U.S. Pub. No. 2013/0103162 to Kieran Costello; and U.S. Pat. No. 7,648,727 to Hossainy et al.. This prior art is representative of coated stent devices.

None of the above inventions and patents, taken either singly or in combination, is seen to describe the invention as claimed. Thus, a need exists for a reliable silicon-reinforced stent with a coated stent structured to maintain a firmer and longer lasting stent and to avoid the above-mentioned problems.

### BRIEF SUMMARY OF THE INVENTION

In view of the foregoing disadvantages inherent in the known stent device art, the presently claimed invention provides a novel reinforced vein stent with a silicon coating. The general purpose of the presently claimed invention, which will be described subsequently in greater detail, is to provide coated stent to maintain a firmer and longer lasting stent shape.

The stent body is an expandable metal mesh. The silicon coating not only reinforces the stent, ensuring it lasts longer, but it also protects the edges from collapsing. The silicon-reinforcement also increases prevention of leaks and tears in the stent, which can cause serious heart issues. The coating also helps prevent plaque build up on and around the stent.

The present invention holds significant improvements and serves as a silicon reinforced vein stent. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention that are believed to be novel are particularly pointed out and distinctly claimed in the concluding portion of the specification. These and other features, aspects, and advantages of the presently claimed invention will become better understood with reference to the following drawings and detailed description.

### BRIEF DESCRIPTION OF THE DRAWINGS

The figures that accompany the written portion of this specification illustrate embodiments and method(s) of use for the presently claimed invention, Coated Stent, constructed and operative according to the teachings of the presently claimed invention.

FIG. 1 shows a perspective view illustrating a reinforced, coated stent being inserted into a vein with plaque build up, according to an embodiment of the presently claimed invention.

FIG. 2 shows a perspective view illustrating an inserted reinforced, coated stent being inserted into a vein, according to an embodiment of the presently claimed invention.

FIG. 3 shows a perspective view illustrating an inserted reinforced, coated stent after insertion, according to an embodiment of the presently claimed invention.

The various embodiments of the presently claimed invention will hereinafter be described in conjunction with the appended drawings.

### DETAILED DESCRIPTION

As discussed above, embodiments of the presently claimed invention relate to a stent device and more particularly to a silicon-reinforced stent, as used to improve durability of the stent and prevent plaque build up.

Generally, the silicon-reinforced stent comprising a novel product offering consumers a practical solution to the aforementioned challenges. As the name implies, the silicon-reinforced stent is a specially designed stent structured and arranged with a silicon coating over the metal stent structure to maintain a firmer and longer lasting stent. The silicon may be similar to Vaseline and may also prevent plaque from further accumulating in the vein. Additionally, the silicon coating will help prevent the edges of the metal structure from collapsing, thereby ensuring that if the stent moves or comes out, the movement will cause less damage.

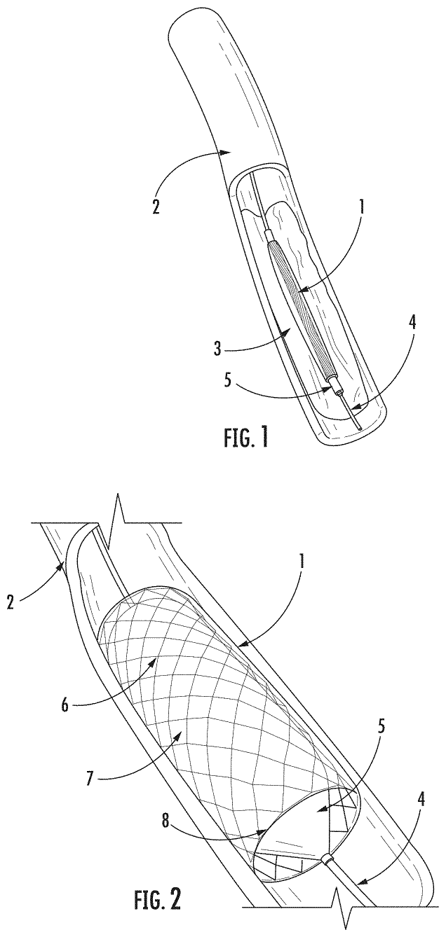

Referring now to the drawing of FIG. 1, the silicon-reinforced stent 1 can be inserted into an area of a vein 2 narrowed by plaque 3 on a guide wire 4 with an inflatable balloon 5 in accordance with standard stent inserting procedures.

As shown in FIG. 2, once inserted in the correct location in the vein 2, the balloon 5 on the guide wire 4 is inflated, expanding both the metal mesh portion 6 and the silicon coating 7 of the stent 1. After the metal mesh portion 6 is expanded, stretching the silicon coating 7, the balloon 5 can be deflated and the guide wire 4 removed from the vein 2.

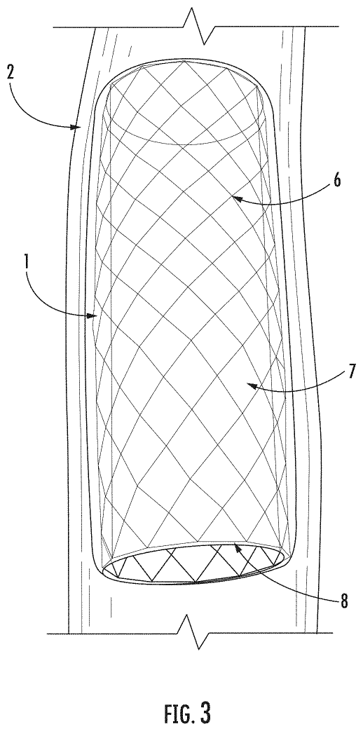

As shown in FIG. 3, the metal mesh portion 6 of the stent 1 remains expanded inside the vein 2 after the balloon 5 and guide wire 4 are removed. The silicon coating 7 wraps around the outside of the metal mesh portion 6. The silicon coating 7 may optionally be additionally adhered to the inside of the metal mesh portion 6. The silicon coating 7 is built up around the lip 8 to protect the edge. This thicker build up of silicon around the lip 8 helps prevent the edges of the metal mesh portion 6 from collapsing, thereby helping to keep the stent 1 in place inside the vein 2.

The metal mesh portion 6 is made from any suitable metal material, such as nitinol. The silicon coating 7 helps to prevent plaque 3 build up inside the stent 1, as silicon has a smoother surface than the metal mesh portion 6, making it more difficult for plaque 3 to adhere to the surface of the stent 1. The silicon-reinforced stent is cost-effective to produce in the embodiments, as shown in FIGS. 1-3.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claim:

1. A silicon-reinforced vein stent comprising:
(a) a metal mesh portion with
(i) an outer side;
(ii) a first end;
(iii) a first lip encompassing the first end;
(iv) a second end; and
(v) a second lip encompassing the second end; and
(b) a silicon coating,
wherein the silicon coating is operably adhered to the outer side of the metal mesh portion, and
wherein the silicon coating covers the first lip and the second lip.

2. The silicon-reinforced vein stent according to claim 1, wherein the metal mesh portion is made of nickel titanium.

3. The silicon-reinforced vein stent according to claim 1, wherein the silicon coating is also operably adhered to an inner side of the metal mesh portion.
